(19) United States
(12) Patent Application Publication
(10) Pub. No.: US 2019/0240431 A1
(43) Pub. Date: Aug. 8, 2019

(54) WEARBALE INTRANASAL CLIP APPARATUS AND METHOD OF USE

(71) Applicant: Nathan T. Prather, Kansas City, MO (US)

(72) Inventor: Nathan T. Prather, Kansas City, MO (US)

(21) Appl. No.: 16/173,819

(22) Filed: Oct. 29, 2018

Related U.S. Application Data
(60) Provisional application No. 62/625,551, filed on Feb. 2, 2018.

(51) Int. Cl. A61M 15/08; A61M 21/02

(52) U.S. Cl. CPC A61M 15/08; A61M 2021/0016; A61M 21/02

(57) ABSTRACT

A wearable intranasal device that dispenses concentrated oils, specifically Aromatherapy and Cannabidiol or CBD, allowing each to evaporate naturally through the nasal cavity providing temporary relief of a variety of symptoms these products have been positively found to impact. The device is a single unit consisting of two barrels on both ends joined by a bridging center arch and which will hug the septum of the user ergonomically not causing any irritation to the user. Each barrel receives a cotton wick which will hold the concentrated oil(s).

### CROSS-REFERENCE TO RELATED APPLICATION

This application claims priority in U.S. Provisional Patent Application No. 62/625,551 Filed Feb. 2, 2018, which is incorporated herein by reference.

### BACKGROUND OF THE INVENTION

### 1. Field of the Invention

The present invention relates generally to a wearable nasal device and method for use thereof, and more specifically to a wearable intranasal device that dispenses concentrated oils.

### 2. Description of the Related Art

Aromatherapy's roots can be traced back more than 3,500 years when the first users burnt aromatic herbs to assist in treatments of their ill or to relax their dying. Since that time, the process has been refined through science to capture the oils of different raw plant materials including but not limited to flowers, fruits, leaves, wood, bark, peel and seeds. The oils are extracted through the processes distillation (the extraction of the essential meaning or most important aspects of something by a process of heating and cooling), hydro distillation (a variant of steam and distillation in which material is soaked for some time in water and after with the mixture is heated and volatile materials are carried away in steam, condensed and separated) and expression (the production of something, especially by pressing or squeezing it out).

Essential Oils have been proven in tests around the world to help manage a variety of symptoms including but not limited to food cravings in overweight recipients; assistance for users who suffer from anxiety feel less anxious, giving them a sense of calmness while providing a more positive outlook; provide users with better memory while increasing alertness; help users ease the pain of headaches and migraines; help users before and after certain surgeries which keeps the amount of analgesics lower than one who does not use; help users increase blood flow as a natural aphrodisiac and provides Athlete users with more energy, motivation, speed and confidence or simply clearing up a running nose. The relief or benefits that have been listed only encompass a few Essential Oils, some such as peppermint have been proven to assist with multiple health related ailments.

Cannabidiol (CBD) roots can be traced back to the 19th century assisting Queen Victoria with her menstrual cramps. This is not to be confused with tetrahydrocannabinol (THC) which provides the “high” sensation; rather, CBD is a chemical compound in the cannabis plant that provides a relaxing state to the user. There are three processes of extraction, C02 extraction (using carbon dioxide under high pressure and extremely low temperatures to isolate, preserve and maintain the purity of the medicinal oil), Ethanol (high grade grain alcohol used to create high quality cannabis oil appropriate for vape pen cartridges and other similar products) and Olive Oil (a safe and inexpensive way to extract cannabis oil but creates a perishable substance). CBD was first successfully found to assist with epilepsy; however, with further research it has been identified as the responsible component for medical marijuana's benefits, including but not limited to; cigarette addiction, acne, diabetes, multiple sclerosis, rheumatoid arthritis and other chronic pain conditions. It assists with these ailments by reducing inflammation, increasing insulin production and regulating the immune system to provide a better way of life for the user. There are advancements being made every day for CBD and as of August 2017, there are no legal barriers that stand in the way of market entry.

Both Essential Oils and Cannabidiol (CBD) have been proven through lab tests, clinical trials and user testimonials to provide a better way of life for the people who use them and though a few products have been patented providing the treatment intranasally; however, what is needed is an improved delivery device for such oils. Creating a wearable intranasal device gives the user the temporary relief from irritating symptoms, ability to provide focus or create mood enhancement and in some cases, an overall better way of life for the people who use them.

Heretofore there has not been available a system or method for a wearable intranasal device with the advantages and features of the present invention.

### BRIEF SUMMARY OF THE INVENTION

The present invention generally provides a wearable intranasal device that dispenses concentrated oils, specifically Aromatherapy and Cannabidiol or CBD, allowing each to evaporate naturally through the nasal cavity providing temporary relief of a variety of symptoms these products have been positively found to impact. The device is a single unit consisting of two barrels on both ends joined by a bridging center arch and which will hug the septum of the user ergonomically not causing any irritation to the user. Each barrel receives a cotton wick which will hold the concentrated oil(s). The barrels include a cap which seals the barrel over the wick. Each barrel will have ventilation holes located at the top and bottom in order to provide superior ventilation, granting outside air to flow around and through the device to lessen the concentration of the oil(s) in the user's nasal cavity.

### BRIEF DESCRIPTION OF THE DRAWINGS

The drawings constitute a part of this specification and include exemplary embodiments of the present invention illustrating various objects and features thereof.

FIG. 1 is a front elevational view of a preferred embodiment of the present invention shown in a typical environment.

FIG. 2 is a three-dimensional isometric view thereof shown in isolation.

FIG. 3 is a three-dimensional isometric view of a bottom portion of the embodiment thereof including bottom barrel portions and bridging arch.

FIG. 4 is a three-dimensional isometric view of a top barrel element thereof.

FIG. 5 is a front elevational view thereof showing the top barrel elements being fitted to the bottom portion thereof.

FIG. 6 is a front elevational view thereof shown in a fully assembled orientation.

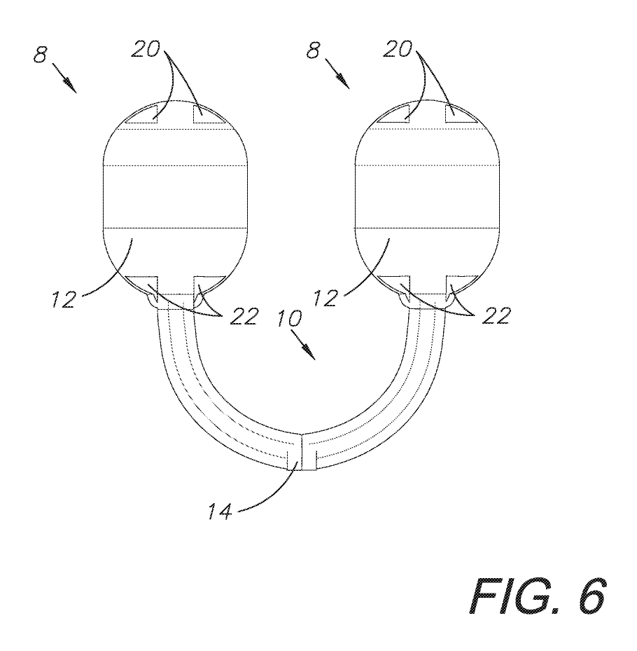

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

### II. Preferred Embodiment Intranasal Device System 2

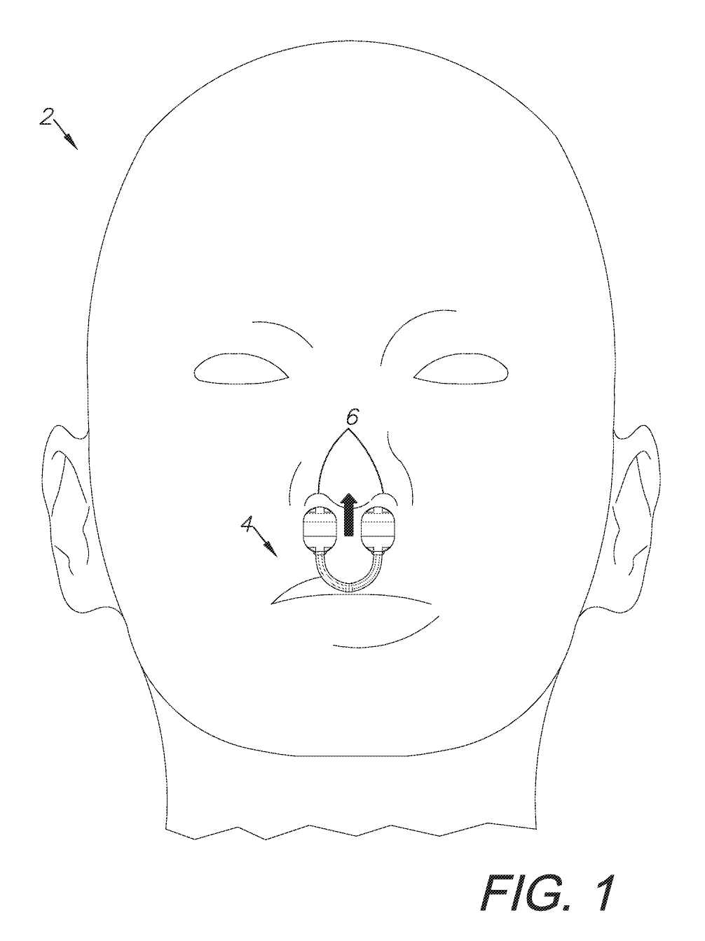

FIG. 1 shows an intranasal device system 2 where a wearable intranasal device 4 is deployed within the nostrils 6 of a user. This allows for the delivery of evaporating oils, such as aromatherapy and/or cannabinol (CBD) oils, into the nasal cavities of the user.

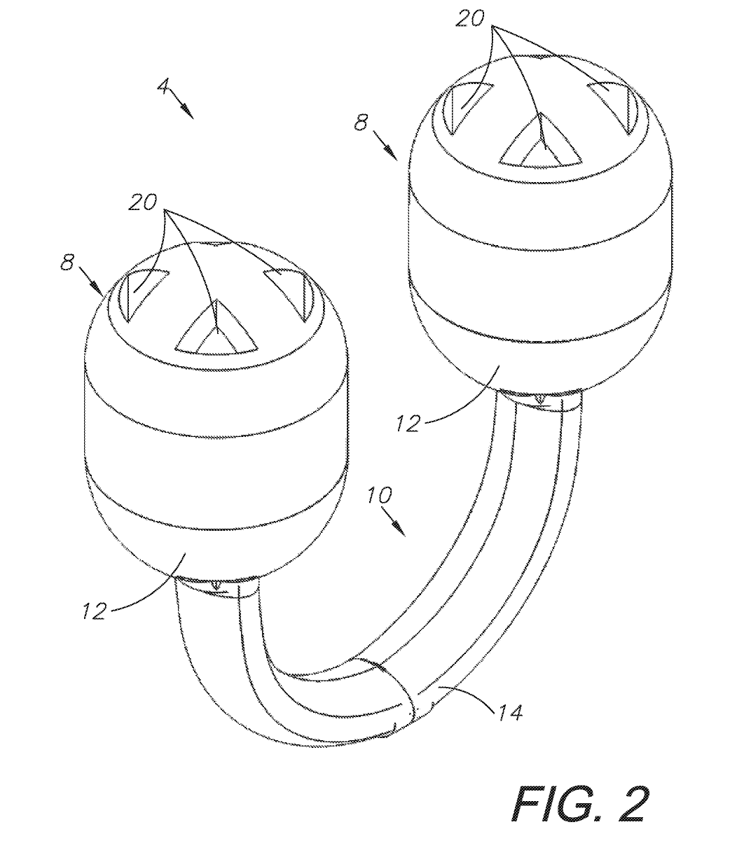

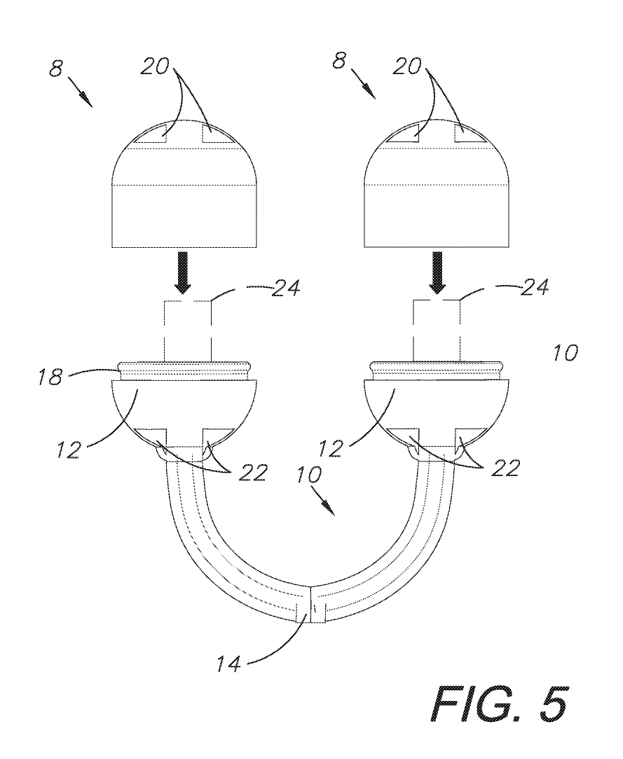

As shown in FIG. 2, the intranasal device 4 generally includes a bottom portion 10 and a pair of top barrel portions 8. The bottom portion 10 includes two bottom barrel portions 12 joined by a bridging arch 14. The top barrel portions 8 include ventilation holes 20 for providing ventilation into the wick 24 stored between the top 8 and bottom 12 barrel portions as shown in FIG. 5. The bottom barrel portions 12 similarly have corresponding ventilation holes 22.

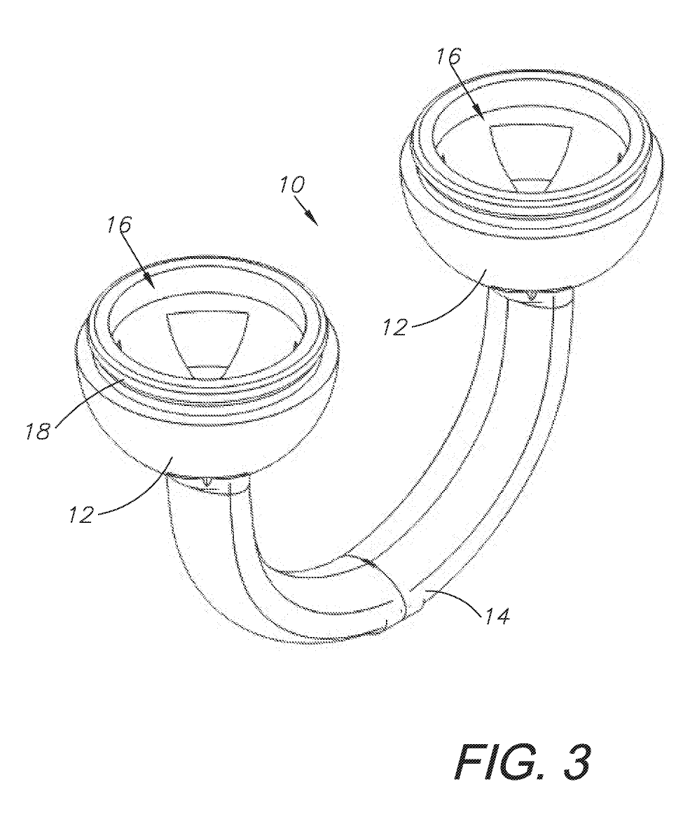

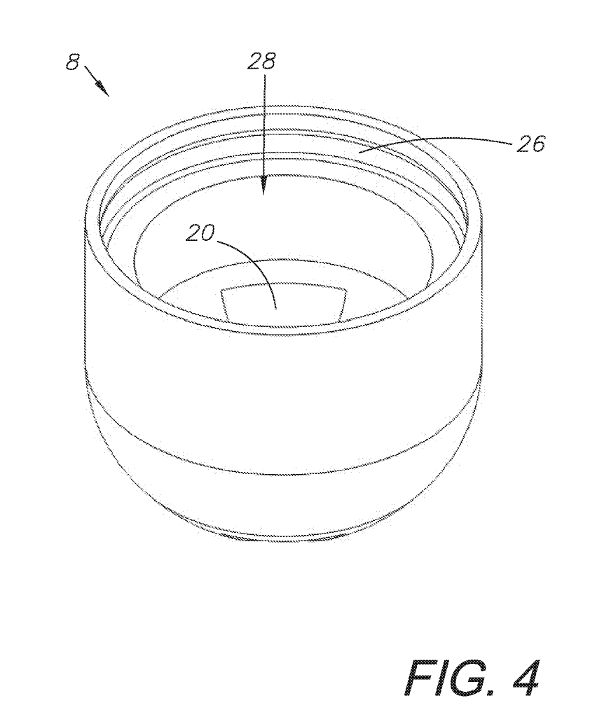

As shown in FIG. 3, the bottom barrel portions 12 include an internal cavity 16 for receiving the wick 24 which contains the oils for delivery to the user. A connector ring 18 allows for the locking onto the bottom barrel portion 12 of the top barrel portion 8 which, as shown in FIG. 4, includes a corresponding connector lip 26. The top barrel portion 8 also includes a corresponding internal cavity 28 for receiving the wick.

The intranasal device 4 is preferably molded of bisphenol A-free (BPA-free) plastic or similar resin, a non-irritating plastic that is flexible and non-corrosive with the use of concentrated oils. When worn, very little of the intranasal clip will show. As indicated in FIG. 1 only the arch 14 that joins the two barrels will be visible.

Each of the barrels can be opened to insert aroma wicks containing the oils. The barrels include openings to allow vapors from the oils to be inhaled into the nose. The dosages may vary depending on the requirements of the user.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. A wearable intranasal oil delivery system comprising:
a pair of barrel chambers joined by a bridging arch, each of said barrel chambers configured to be inserted into a cavity;
each of said barrel chambers comprising a top portion and a bottom portion, wherein said top portion of each respective barrel chamber may be removed from said bottom portion;
each of said barrel chambers comprising an internal cavity configured for storing a wick; and
each of said barrel chambers comprising a plurality of ventilation holes for delivering aroma from said wick into said cavity.

2. The system of claim 1, wherein said wick contains oils.

3. The system of claim 2, wherein said oils comprise aromatherapy oils.

4. The system of claim 2, wherein said oils comprise Cannabidiol oils.

5. The system of claim 1, further comprising:
said bottom portion of said barrel chamber comprising a receiver ring;
said top portion of said barrel chamber comprising a locking ring; and
wherein said receiver ring and said locking ring are configured to releasably join said top portion and said bottom portion.
